SEQUENCE LISTING (128 sequences)

SEQ ID NO: 1 - DNA, 37 bp, Artificial Sequence
  synthetic_primer
tagatcgaaaggtgcacaaagatgaaaccgaccacca

SEQ ID NO: 2 - DNA, 41 bp, Artificial Sequence
  synthetic_primer
ccgctctagaactagtggatcttaatggaaactgtgttctt

SEQ ID NO: 3 - DNA, 36 bp, Artificial Sequence
  synthetic_primer
tagatcgaaaggtgcacaaagatgaaaccgaccacc

SEQ ID NO: 4 - DNA, 40 bp, Artificial Sequence
  synthetic_primer
ccgctctagaactagtggatcttaatggaaactgtgttct

SEQ ID NO: 5 - DNA, 38 bp, Artificial Sequence
  synthetic_primer
tagatcgaaaggtgcacaaagatgaaaccaaccaccat

SEQ ID NO: 6 - DNA, 41 bp, Artificial Sequence; identical to SEQ ID NO: 2
  synthetic_primer

SEQ ID NO: 7 - DNA, 42 bp, Artificial Sequence
  synthetic_primer
tagatcgaaaggtgcacaaagatgtcattaaagcaaataact

SEQ ID NO: 8 - DNA, 41 bp, Artificial Sequence
  synthetic_primer
ccgctctagaactagtggatcttattggaaactgtgttctt

SEQ ID NO: 9 - DNA, 36 bp, Artificial Sequence
  synthetic_primer
tagatcgaaaggtgcacaaagatgaaacccaccacc

SEQ ID NO: 10 - DNA, 39 bp, Artificial Sequence
  synthetic_primer
ccgctctagaactagtggatcttagttaaatgagtgctc

SEQ ID NO: 11 - DNA, 37 bp, Artificial Sequence; identical to SEQ ID NO: 1
  synthetic_primer

SEQ ID NO: 12 - DNA, 40 bp, Artificial Sequence; identical to SEQ ID NO: 4
  synthetic_primer

SEQ ID NO: 13 - DNA, 36 bp, Artificial Sequence
  synthetic_primer
tagatcgaaaggtgcacaaagatgaaacccaccacg

SEQ ID NO: 14 - DNA, 38 bp, Artificial Sequence
  synthetic_primer
ccgctctagaactagtggatcttactggaaactgtgct

SEQ ID NO: 15 - DNA, 44 bp, Artificial Sequence
  synthetic_primer
tagatcgaaaggtgcacaaagatgaaaactttaaatcatttaaa

SEQ ID NO: 16 - DNA, 39 bp, Artificial Sequence
  synthetic_primer
ccgctctagaactagtggatcttagtcatgttgctctac

SEQ ID NO: 17 - DNA, 37 bp, Artificial Sequence
  synthetic_primer
tagatcgaaaggtgcacaaagatgccagatgtaaccg

SEQ ID NO: 18 - DNA, 36 bp, Artificial Sequence
  synthetic_primer
ccgctctagaactagtggatctcatgcggagaaccc

SEQ ID NO: 19 - DNA, 36 bp, Artificial Sequence
  synthetic_primer
tagatcgaaaggtgcacaaagatgcctgatgtgacc

SEQ ID NO: 20 - DNA, 36 bp, Artificial Sequence
  synthetic_primer
ccgctctagaactagtggatctcatgcggagaatcc

SEQ ID NO: 21 - DNA, 39 bp, Artificial Sequence
  synthetic_primer
tagatcgaaaggtgcacaaagatgtcggtacataaagaa

SEQ ID NO: 22 - DNA, 42 bp, Artificial Sequence
  synthetic_primer
ccgctctagaactagtggatcttaatattgttctttttcgtt

SEQ ID NO: 23 - DNA, 36 bp, Artificial Sequence; identical to SEQ ID NO: 9
  synthetic_primer

SEQ ID NO: 24 - DNA, 40 bp, Artificial Sequence
  synthetic_primer
ccgctctagaactagtggatcttagttgaaagagtgctct

SEQ ID NO: 25 - DNA, 40 bp, Artificial Sequence
  synthetic_primer
tagatcgaaaggtgcacaaagatgtctacagcaaaaaaag

SEQ ID NO: 26 - DNA, 40 bp, Artificial Sequence
  synthetic_primer
ccgctctagaactagtggatcttaatactgttcatcgtca

SEQ ID NO: 27 - DNA, 41 bp, Artificial Sequence
  synthetic_primer
cttgatatcgaattcctgcattcagggtagttgactaaaga

SEQ ID NO: 28 - DNA, 17 bp, Artificial Sequence
  synthetic_primer
ctttgtgcacctttcga

SEQ ID NO: 29 - DNA, 40 bp, Artificial Sequence
  synthetic_primer
tgaattcgagctcggtacccgaaatagcgcttgatgaatc

SEQ ID NO: 30 - DNA, 40 bp, Artificial Sequence
  synthetic_primer
ggttgctacctgcacccggggggcatgagtatagatgtga

SEQ ID NO: 31 - DNA, 40 bp, Artificial Sequence
  synthetic_primer
ctatactcatgccccccgggtgcaggtagcaaccacaaag

SEQ ID NO: 32 - DNA, 40 bp, Artificial Sequence
  synthetic_primer
gtcgactctagaggatcccctatgtggcgttgggtgcagc

SEQ ID NO: 33 - DNA, 40 bp, Artificial Sequence
  synthetic_primer
catctatactcatgccccccttcagggtagttgactaaag

SEQ ID NO: 34 - DNA, 40 bp, Artificial Sequence
  synthetic_primer
tgtggttgctacctgcacccttaatggaaactgtgttctt

SEQ ID NO: 35 - DNA, 20 bp, Artificial Sequence
  synthetic_primer
cccacccggtgtcattcgac

SEQ ID NO: 36 - DNA, 19 bp, Artificial Sequence
  synthetic_primer
gcgcatccagctcatcggt

SEQ ID NO: 37 - protein, 264 aa, Artificial Sequence
  synthetic_PanB WT
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 38 - DNA, 40 bp, Artificial Sequence
  synthetic_primer
gtttctaccagccactcggagccttcaattttgaccatgt

SEQ ID NO: 39 - DNA, 40 bp, Artificial Sequence
  synthetic_primer
acatggtcaaaattgaaggctccgagtggctggtagaaac

SEQ ID NO: 40 - DNA, 40 bp, Artificial Sequence
  synthetic_primer
gtttctaccagccactcgcagccttcaattttgaccatgt

SEQ ID NO: 41 - DNA, 40 bp, Artificial Sequence
  synthetic_primer
acatggtcaaaattgaaggctgcgagtggctggtagaaac

SEQ ID NO: 42 - DNA, 40 bp, Artificial Sequence
  synthetic_primer
gtttctaccagccactccaggccttcaattttgaccatgt

SEQ ID NO: 43 - DNA, 40 bp, Artificial Sequence
  synthetic_primer
acatggtcaaaattgaaggcctggagtggctggtagaaac

SEQ ID NO: 44 - DNA, 40 bp, Artificial Sequence
  synthetic_primer
gtttctaccagccactcgatgccttcaattttgaccatgt

SEQ ID NO: 45 - DNA, 40 bp, Artificial Sequence
  synthetic_primer
acatggtcaaaattgaaggcatcgagtggctggtagaaac

SEQ ID NO: 46 - DNA, 40 bp, Artificial Sequence
  synthetic_primer
gtttctaccagccactcggtgccttcaattttgaccatgt

SEQ ID NO: 47 - DNA, 40 bp, Artificial Sequence
  synthetic_primer
acatggtcaaaattgaaggcaccgagtggctggtagaaac

SEQ ID NO: 48 - DNA, 40 bp, Artificial Sequence
  synthetic_primer
gtttctaccagccactccacgccttcaattttgaccatgt

SEQ ID NO: 49 - DNA, 40 bp, Artificial Sequence
  synthetic_primer
acatggtcaaaattgaaggcgtggagtggctggtagaaac

SEQ ID NO: 50 - DNA, 40 bp, Artificial Sequence
  synthetic_primer
gtttctaccagccactccatgccttcaattttgaccatgt

SEQ ID NO: 51 - DNA, 40 bp, Artificial Sequence
  synthetic_primer
acatggtcaaaattgaaggcatggagtggctggtagaaac

SEQ ID NO: 52 - DNA, 40 bp, Artificial Sequence
  synthetic_primer
gtttctaccagccactcatcgccttcaattttgaccatgt

SEQ ID NO: 53 - DNA, 40 bp, Artificial Sequence
  synthetic_primer
acatggtcaaaattgaaggcgatgagtggctggtagaaac

SEQ ID NO: 54 - DNA, 40 bp, Artificial Sequence
  synthetic_primer
gtttctaccagccactcttcgccttcaattttgaccatgt

SEQ ID NO: 55 - DNA, 40 bp, Artificial Sequence
  synthetic_primer
acatggtcaaaattgaaggcgaagagtggctggtagaaac

SEQ ID NO: 56 - DNA, 40 bp, Artificial Sequence
  synthetic_primer
gtttctaccagccactcgttgccttcaattttgaccatgt

SEQ ID NO: 57 - DNA, 40 bp, Artificial Sequence
  synthetic_primer
acatggtcaaaattgaaggcaacgagtggctggtagaaac

SEQ ID NO: 58 - DNA, 40 bp, Artificial Sequence
  synthetic_primer
gtttctaccagccactcctggccttcaattttgaccatgt

SEQ ID NO: 59 - DNA, 40 bp, Artificial Sequence
  synthetic_primer
acatggtcaaaattgaaggccaggagtggctggtagaaac

SEQ ID NO: 60 - DNA, 40 bp, Artificial Sequence
  synthetic_primer
gtttctaccagccactctgcgccttcaattttgaccatgt

SEQ ID NO: 61 - DNA, 40 bp, Artificial Sequence
  synthetic_primer
acatggtcaaaattgaaggcgcagagtggctggtagaaac

SEQ ID NO: 62 - protein, 264 aa, Artificial Sequence
  synthetic_PanB with G116A mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGAEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 63 - protein, 264 aa, Artificial Sequence
  synthetic_PanB with G116N mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGNEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 64 - protein, 264 aa, Artificial Sequence
  synthetic_PanB with G116T mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGTEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 65 - protein, 264 aa, Artificial Sequence
  synthetic_PanB with G116E mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGEEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 66 - protein, 264 aa, Artificial Sequence
  synthetic_PanB with G116S mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGSEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 67 - protein, 264 aa, Artificial Sequence
  synthetic_PanB with G116V mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGVEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 68 - protein, 264 aa, Artificial Sequence
  synthetic_PanB with G116I mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGIEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 69 - protein, 264 aa, Artificial Sequence
  synthetic_PanB with G116L mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGLEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 70 - protein, 264 aa, Artificial Sequence
  synthetic_PanB with G116D mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGDEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 71 - protein, 264 aa, Artificial Sequence
  synthetic_PanB with G116C mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGCEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 72 - protein, 264 aa, Artificial Sequence
  synthetic_PanB with G116Q mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGQEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 73 - protein, 264 aa, Artificial Sequence
  synthetic_PanB with G116M mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGMEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 74 - DNA, 40 bp, Artificial Sequence
  primer
agatcgaaaggtgcacaaagatgaaaccgaccaccatctc

SEQ ID NO: 75 - DNA, 40 bp, Artificial Sequence
  primer
aggggcgcggcgatgaacgcggcgatcaactgctcagcga

SEQ ID NO: 76 - DNA, 40 bp, Artificial Sequence
  primer
ctgagcagttgatcgccgcgttcatcgccgcgcccctgaa

SEQ ID NO: 77 - DNA, 40 bp, Artificial Sequence
  primer
cgctctagaactagtggatcttaatggaaactgtgttctt

SEQ ID NO: 78 - DNA, 40 bp, Artificial Sequence
  primer
aggggcgcggcgatgaatccggcgatcaactgctcagcga

SEQ ID NO: 79 - DNA, 40 bp, Artificial Sequence
  primer
ctgagcagttgatcgccggattcatcgccgcgcccctgaa

SEQ ID NO: 80 - DNA, 40 bp, Artificial Sequence
  primer
aggggcgcggcgatgaatacggcgatcaactgctcagcga

SEQ ID NO: 81 - DNA, 40 bp, Artificial Sequence
  primer
ctgagcagttgatcgccgtattcatcgccgcgcccctgaa

SEQ ID NO: 82 - DNA, 40 bp, Artificial Sequence
  primer
aggggcgcggcgatgaatgcggcgatcaactgctcagcga

SEQ ID NO: 83 - DNA, 40 bp, Artificial Sequence
  primer
ctgagcagttgatcgccgcattcatcgccgcgcccctgaa

SEQ ID NO: 84 - DNA, 40 bp, Artificial Sequence
  primer
aggggcgcggcgatgaaccaggcgatcaactgctcagcga

SEQ ID NO: 85 - DNA, 40 bp, Artificial Sequence
  primer
ctgagcagttgatcgcctggttcatcgccgcgcccctgaa

SEQ ID NO: 86 - DNA, 40 bp, Artificial Sequence
  primer
aggggcgcggcgatgaacacggcgatcaactgctcagcga

SEQ ID NO: 87 - DNA, 40 bp, Artificial Sequence
  primer
ctgagcagttgatcgccgtgttcatcgccgcgcccctgaa

SEQ ID NO: 88 - DNA, 40 bp, Artificial Sequence
  primer
aggggcgcggcgatgaactgggcgatcaactgctcagcga

SEQ ID NO: 89 - DNA, 40 bp, Artificial Sequence
  primer
ctgagcagttgatcgcccagttcatcgccgcgcccctgaa

SEQ ID NO: 90 - DNA, 40 bp, Artificial Sequence
  primer
aggggcgcggcgatgaaatcggcgatcaactgctcagcga

SEQ ID NO: 91 - DNA, 40 bp, Artificial Sequence
  primer
ctgagcagttgatcgccgatttcatcgccgcgcccctgaa

SEQ ID NO: 92 - DNA, 40 bp, Artificial Sequence
  primer
aggggcgcggcgatgaaaccggcgatcaactgctcagcga

SEQ ID NO: 93 - DNA, 40 bp, Artificial Sequence
  primer
ctgagcagttgatcgccggtttcatcgccgcgcccctgaa

SEQ ID NO: 94 - DNA, 40 bp, Artificial Sequence
  primer
aggggcgcggcgatgaaaagggcgatcaactgctcagcga

SEQ ID NO: 95 - DNA, 40 bp, Artificial Sequence
  primer
ctgagcagttgatcgcccttttcatcgccgcgcccctgaa

SEQ ID NO: 96 - DNA, 40 bp, Artificial Sequence
  primer
aggggcgcggcgatgaagtgggcgatcaactgctcagcga

SEQ ID NO: 97 - DNA, 40 bp, Artificial Sequence
  primer
ctgagcagttgatcgcccacttcatcgccgcgcccctgaa

SEQ ID NO: 98 - DNA, 40 bp, Artificial Sequence
  primer
aggggcgcggcgatgaaatgggcgatcaactgctcagcga

SEQ ID NO: 99 - DNA, 40 bp, Artificial Sequence
  primer
ctgagcagttgatcgcccatttcatcgccgcgcccctgaa

SEQ ID NO: 100 - DNA, 40 bp, Artificial Sequence
  primer
aggggcgcggcgatgaagatggcgatcaactgctcagcga

SEQ ID NO: 101 - DNA, 40 bp, Artificial Sequence
  primer
ctgagcagttgatcgccatcttcatcgccgcgcccctgaa

SEQ ID NO: 102 - DNA, 40 bp, Artificial Sequence
  primer
aggggcgcggcgatgaacagggcgatcaactgctcagcga

SEQ ID NO: 103 - DNA, 40 bp, Artificial Sequence
  primer
ctgagcagttgatcgccctgttcatcgccgcgcccctgaa

SEQ ID NO: 104 - DNA, 40 bp, Artificial Sequence
  primer
aggggcgcggcgatgaaaacggcgatcaactgctcagcga

SEQ ID NO: 105 - DNA, 40 bp, Artificial Sequence
  primer
ctgagcagttgatcgccgttttcatcgccgcgcccctgaa

SEQ ID NO: 106 - DNA, 40 bp, Artificial Sequence; identical to SEQ ID NO: 102
  primer

SEQ ID NO: 107 - DNA, 40 bp, Artificial Sequence; identical to SEQ ID NO: 103
  primer

SEQ ID NO: 108 - DNA, 40 bp, Artificial Sequence
  primer
cttgatatcgaattcctgcattcagggtagttgactaaag

SEQ ID NO: 109 - DNA, 40 bp, Artificial Sequence
  primer
gagatggtggtcggtttcatctttgtgcacctttcgatct

SEQ ID NO: 110 - protein, 264 aa, Artificial Sequence
  PanB with A159L mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDELGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 111 - protein, 264 aa, Artificial Sequence
  PanB with A159R mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDERGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 112 - protein, 264 aa, Artificial Sequence
  PanB with A159S mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDESGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 113 - protein, 264 aa, Artificial Sequence
  PanB with A159Y mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEYGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 114 - protein, 264 aa, Artificial Sequence
  PanB with A159C mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDECGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 115 - protein, 264 aa, Artificial Sequence
  PanB with A159P mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEPGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 116 - protein, 264 aa, Artificial Sequence
  PanB with A159H mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEHGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 117 - protein, 264 aa, Artificial Sequence
  PanB with A159I mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEIGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 118 - protein, 264 aa, Artificial Sequence
  PanB with A159T mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDETGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 119 - protein, 264 aa, Artificial Sequence
  PanB with A159K mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEKGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 120 - protein, 264 aa, Artificial Sequence
  PanB with A159V mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEVGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 121 - protein, 264 aa, Artificial Sequence
  PanB with A159M mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEMGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 122 - protein, 264 aa, Artificial Sequence
  PanB with A159D mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEDGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 123 - protein, 264 aa, Artificial Sequence
  PanB with A159E mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEEGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 124 - protein, 264 aa, Artificial Sequence
  PanB with A159N mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDENGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 125 - protein, 264 aa, Artificial Sequence
  PanB with A159Q mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEQGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 126 - DNA, 40 bp, Artificial Sequence
  primer
gtttctaccagccactcggcgccttcaattttgaccatgt

SEQ ID NO: 127 - DNA, 40 bp, Artificial Sequence
  primer
tggtcaaaattgaaggcgccgagtggctggtagaaaccgt

SEQ ID NO: 128 - protein, 264 aa, Artificial Sequence
  PanB with G116A and A159L mutation
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGAEWLVETVQMLTERAVPVCGHLGLTPQSVNIFGGYKVQGRGDELGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

(19) United States
(12) Patent Application Publication
(10) Pub. No.: US 2025/0290050 A1
(43) Pub. Date: Sep. 18, 2025

(54) MICROORGANISM HAVING ENHANCED ACTIVITY OF 3-METHYL-2-OXOBUTANOATE HYDROXYMETHYLTRANSFERASE AND USES THEREOF

(71) Applicant: CJ CheilJedang Corporation, Seoul (KR)

(72) Inventors: Yee-Seul SO, Seoul (KR); Kwang Soo SHIN, Seoul (KR); Jihyun SHIM, Seoul (KR); Kwang Woo LEE, Seoul (KR); Yeon-Jae JANG, Seoul (KR)

(21) Appl. No.: 17/776,363

(22) PCT Filed: Mar. 18, 2022
(86) PCT No.: PCT/KR2022/003845

§ 371 Date: May 12, 2022

(30) Foreign Application Priority Data
May 10, 2021 (KR) 10-2021-0060009

(51) Int. Cl. C12N 9/10; C12N 15/77; C12P 7/42; C12P 13/02; C12R 1/15

(52) U.S. Cl. CPC C12N 9/1014; C12N 15/77; C12P 7/42; C12P 13/02; C12R 2001/15; C12Y 201/02011

(57) ABSTRACT

A 3-methyl-2-oxobutanoate hydroxymethyltransferase variant, a microorganism having enhanced activity of 3-methyl-2-oxobutanoate hydroxymethyltransferase, a composition for producing pantothenic acid and/or pantoic acid comprising the microorganism, and a method for preparing pantothenic acid and/or pantoic acid comprising culturing the microorganism are provided.

### TECHNICAL FIELD

### Cross-Reference to Related Application(S)

The present application claims the priority based on Korean Patent Application No. 10-2021-0060009 filed on May 10, 2021, and all the contents disclosed in the document of the corresponding Korean Patent Application are incorporated by reference as a part of the present description.

A 3-methyl-2-oxobutanoate hydroxymethyltransferase variant, a microorganism having enhanced activity of 3-methyl-2-oxobutanoate hydroxymethyltransferase, a composition for producing pantothenic acid and/or pantoic acid comprising the microorganism, and a method for preparing pantothenic acid and/or pantoic acid comprising culturing the microorganism are provided.

### BACKGROUND ART

Pantothenic acid is a substance belonging to the vitamin B complex, also called vitamin B5, and is one of commercially important substances that are variously applied to cosmetics, medicines, human nutrition, animal nutrition, and the like. Pantothenic acid is a structure in which beta-alanine is linked to pantoic acid by an amide bond.

Pantothenic acid or pantoic acid may be prepared by chemical synthesis, or may be prepared biotechnologically by fermenting an appropriate microorganism in a suitable medium. An advantage of the biotechnological preparation method using a microorganism is that a desired stereo-isomeric D-form of pantothenic acid or pantoic acid is formed.

Accordingly, it is required to develop a microorganism having an advantageous effect in biotechnologically preparing pantothenic acid and/or pantoic acid and a technology for preparing pantothenic acid and/or pantoic acid with high efficiency using the same.

### PRIOR ART

### Patent Document

(Prior document 1) U.S. Registered U.S. Pat. No. 7,718,205

### DISCLOSURE

### Technical Problem

One example of the present application provides a polypeptide having 3-methyl-2-oxobutanoate hydroxymethyltransferase activity. In one embodiment, (1) the polypeptide may comprise the amino acid sequence in which an amino acid corresponding to the 159th residue from the N-terminus in the amino acid sequence of SEQ ID NO: 37 is substituted with another amino acid. In another embodiment, (2) the polypeptide may comprise the amino acid sequence in which an amino acid corresponding to the 159th residue from the N-terminus is substituted with another amino acid and the 116th residue from the N-terminus is substituted with another amino acid in the amino acid sequence of SEQ ID NO: 37. Counting amino acids from the N-terminus in the amino acid sequence as described above may mean counting with methionine (Met, M) translated from the initiation codon as the first amino acid.

Another example, provides a polynucleotide encoding the polypeptide.

Other example, provides a recombinant vector comprising the polynucleotide. The recombinant vector, may be an expression vector.

Other example provides a microorganism producing pantothenic acid or pantoic acid, which comprises one or more (one, two or all) selected from the group consisting of the polypeptide, polynucleotide encoding the polypeptide, and recombinant vector comprising the polynucleotide, and has enhanced activity of 3-methyl-2-oxobutanoate hydroxymethyltransferase.

The microorganism, may comprise
(1) one or more (one, two or all) selected from the group consisting of a polypeptide in which an amino acid corresponding to the 159th residue from the N-terminus in the amino acid sequence of SEQ ID NO: 37 is substituted with another amino acid, a polynucleotide encoding the polypeptide and a recombinant vector comprising the polynucleotide; or
(2) (1) above, and one or more (one, two or all) selected from the group consisting of a polypeptide in which an amino acid corresponding to the 116th residue from the N-terminus in the amino acid sequence of SEQ ID NO: 37 is substituted with another amino acid, a polynucleotide encoding the polypeptide and a recombinant vector comprising the polynucleotide.

The microorganism may be a genus Corynebacterium microorganism or a genus Escherichia microorganism. The genus Corynebacterium microorganism may be Corynebacterium glutamicum.

Other example, provides a composition for producing pantothenic acid or pantoic acid, comprising the microorganism.

Other example, provides a use of the microorganism in production of pantothenic acid and/or pantoic acid.

Other example, provides a method for preparation of pantothenic acid or pantoic acid, comprising culturing the microorganism in a medium. The method for preparation may further comprise recovering pantothenic acid or pantoic acid from the cultured microorganism, medium or both of them, after the culturing.

### Technical Solution

The present description is to provide a recombinant strain with excellent production ability of pantothenic acid and/or pantoic acid, by searching 3-methyl-2-oxobutanoate hydroxymethyltransferase or its variant which can enhance the production ability of pantothenic acid and/or pantoic acid and introducing this to a microorganism.

In the present description, it was confirmed that the microorganism expressing 3-methyl-2-oxobutanoate hydroxymethyltransferase had excellent production ability of pantothenic acid, and it was confirmed that the production ability of pantothenic acid was further increased, when an amino acid substitution mutation was introduced to a specific position of 3-methyl-2-oxobutanoate hydroxymethyltransferase.

One example provides a polypeptide having 3-methyl-2-oxobutanoate hydroxymethyltransferase activity. In one embodiment, the polypeptide may comprise the amino acid sequence in which an amino acid corresponding to the 159th residue from the N-terminus in the amino acid sequence of SEQ ID NO: 37 is substituted with another amino acid. In another embodiment, the polypeptide may comprise the amino acid sequence in which an amino acid corresponding to the 159th residue from the N-terminus is substituted with another amino acid and an amino acid corresponding to the 116th residue from the N-terminus is substituted with another amino acid in the amino acid sequence of SEQ ID NO: 37. Counting amino acids from the N-terminus in the amino acid sequence as described above may mean counting with methionine (Met, M) translated from the initiation codon as the first amino acid.

Another example, provides a polynucleotide encoding the polypeptide.

Other example, provides a recombinant vector comprising the polynucleotide. The recombinant vector, may be used as an expression vector of the polypeptide.

Other example provides a microorganism with enhanced activity of 3-methyl-2-oxobutanoate hydroxymethyltransferase. The microorganism may be a microorganism producing pantothenic acid or pantoic acid.

That the activity of 3-methyl-2-oxobutanoate hydroxymethyltransferase is enhanced, may be enhanced by that microbial endogenous 3-methyl-2-oxobutanoate hydroxymethyltransferase is mutated or exogenous 3-methyl-2-oxobutanoate hydroxymethyltransferase or variant thereof is introduced.

In one embodiment, the 3-methyl-2-oxobutanoate hydroxymethyltransferase with enhanced activity may comprise the amino acid sequence in which an amino acid corresponding to the 159th residue from the N-terminus in the amino acid sequence of SEQ ID NO: 37 is substituted with another amino acid. Otherwise, the 3-methyl-2-oxobutanoate hydroxymethyltransferase with enhanced activity may comprise the amino acid sequence in which an amino acid corresponding to the 159th residue from the N-terminus is substituted with another amino acid, and an amino acid corresponding to the 116th residue from the N-terminus is substituted with another amino acid in the amino acid sequence of SEQ ID NO: 37.

The microorganism with enhanced activity of 3-methyl-2-oxobutanoate hydroxymethyltransferase, may comprise,
(1) one or more selected from the group consisting of a polypeptide in which an amino acid corresponding to the 159th residue from the N-terminus in the amino acid sequence of SEQ ID NO: 37 is substituted with another amino acid, a polynucleotide encoding the polypeptide and a recombinant vector comprising the polynucleotide; or
(2) (1) above, and one or more selected from the group consisting of a polypeptide in which an amino acid corresponding to the 116th residue from the N-terminus in the amino acid sequence of SEQ ID NO: 37 is substituted with another amino acid, a polynucleotide encoding the polypeptide and a recombinant vector comprising the polynucleotide.

The microorganism with enhanced activity of 3-methyl-2-oxobutanoate hydroxymethyltransferase, may have higher production ability of pantothenic acid and/or pantoic acid, compared to a homogeneous microorganism in which 3-methyl-2-oxobutanoate hydroxymethyltransferase is not enhanced (for example, an amino acid corresponding to the 159th residue and/or an amino acid corresponding to the 116th residue from the N-terminus in the amino acid sequence of SEQ ID NO: 37 is not substituted with another amino acid).

Other example provides a composition for producing pantothenic acid and/or pantoic acid comprising the microorganism with enhanced activity of 3-methyl-2-oxobutanoate hydroxymethyltransferase.

Other example provides a method for producing pantothenic acid and/or pantoic acid comprising culturing the microorganism with enhanced activity of 3-methyl-2-oxobutanoate hydroxymethyltransferase.

Hereinafter, it will be described in more detail.

### Polypeptide

In the present description, pantothenic acid (e.g., D-pantothenic acid) is a compound having the structure of Chemical formula 1, and is a vitamin (vitamin B5) in which B-alanine is linked to pantoic acid by an amide bond, and is a component of coenzyme A (CoA) and acyl carrier protein (ACP), and is involved in various metabolic processes of living organisms.

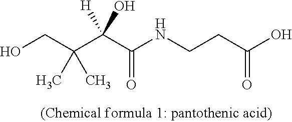

Pantoic acid (e.g., D-pantoic acid) is a compound having the structure of Chemical formula 2, and is a component of various biological activity compounds:

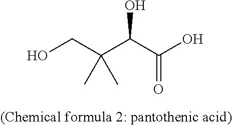

In the present description, 3-methyl-2-oxobutanoate hydroxymethyltransferase is an enzyme catalyzing a process of biosynthesizing tetrahydrofolate and 2-dihydropantoate from 5,10-methylenetetrahydrofolate, 3-methyl-2-oxobutanoate, and water.

In one embodiment, the 3-methyl-2-oxobutanoate hydroxymethyltransferase may be variant in which a mutation in which one or more amino acid residues are substituted, deleted or inserted is introduced.

In one example, the variant of 3-methyl-2-oxobutanoate hydroxymethyltransferase may be one in which an amino acid corresponding to the 159th residue of the amino acid sequence of SEQ ID NO: 37 is substituted with an amino acid, that is an amino acid which is selected from the group consisting of arginine (R), histidine (H), lysine (K), aspartic acid (D), glutamic acid (E), serine(S), threonine (T), asparagine (N), glutamine (Q), cysteine (C), proline (P), valine (V), isoleucine (I), leucine (L), methionine (M), tyrosine (Y), phenylalanine (F), tryptophane (W), and glycine (G) and is different from the original amino acid. In one embodiment, the 3-methyl-2-oxobutanoate hydroxymethyltransferase variant may be one in which an amino acid corresponding to the 159th residue in the amino acid sequence of SEQ ID NO: 37 is substituted with another amino acid, that is, arginine (R), histidine (H), lysine (K), aspartic acid (D), glutamic acid (E), serine(S), threonine (T), asparagine (N), glutamine (Q), cysteine (C), proline (P), valine (V), isoleucine (I), leucine (L), methionine (M) or tyrosine (Y). It is obvious that among the variants, even if some amino acid sequences except for the amino acid corresponding to the 159th residue in the amino acid sequence of SEQ ID NO: 37 are deleted, modified, substituted or added, as long as they show the activity of 3-methyl-2-oxobutanoate hydroxymethyltransferase, they may be included in the variant of the present application.

In addition, in one example, the variant may comprise a polypeptide in which an amino acid corresponding to the 159th residue of the amino acid sequence of SEQ ID NO: 37 in the amino acid sequence having homology or identity of at least 80%, 85%, 90%, 91%, 92%, 93%, 94%, 95%, 96%, 97%, 98%, or 99% or more, to the amino acid sequence disclosed as SEQ ID NO: 37 is substituted with another amino acid. In other words, a polypeptide which comprises a substitution at the position corresponding to the 159th residue of the amino acid sequence of SEQ ID NO: 37, has sequence homology or identity of at least 80%, 90%, 95%, 96%, 97%, 98%, or 99% or more and less than 100%, to the amino acid sequence of SEQ ID NO: 37, and has 3-methyl-2-oxobutanoate hydroxymethyltransferase activity may be included in the variant of the present application.

In one embodiment, the 3-methyl-2-oxobutanoate hydroxymethyltransferase variant may comprise an amino acid sequence selected from SEQ ID NO: 110 to SEQ ID NO: 125, but not limited thereto. It is obvious that even if some amino acid sequences except for the amino acid corresponding to the 159th residue are deleted, modified, substituted or added in the variant consisting of any one sequence selected from SEQ ID NO: 110 to SEQ ID NO: 125, as long as they show the activity of 3-methyl-2-oxobutanoate hydroxymethyltransferase, they may be included in the variant of the present application. In addition, in one example, the variant may comprise a polypeptide having homology or identity of at least 80%, 85%, 90%, 91%, 92%, 93%, 94%, 95%, 96%, 97%, 98%, or 99% or more to any one amino acid sequence selected from SEQ ID NO: 110 to SEQ ID NO: 125 in which an amino acid corresponding to the 159th residue of the amino acid sequence of SEQ ID NO: 37 is fixed. In other words, a polypeptide which comprises a substitution with another amino acid at a position corresponding to the 159th residue of the amino acid sequence of SEQ ID NO: 37 and has sequence homology or identity of at least 80%, 90%, 95%, 96%, 97%, 98%, or 99% or more and less than 100% to any one amino acid sequence selected from SEQ ID NO: 110 to SEQ ID NO: 125 and has 3-methyl-2-oxobutanoate hydroxymethyltransferase activity may be included in the variant of the present application.

In one example, in the variant of 3-methyl-2-oxobutanoate hydroxymethyltransferase,
(1) an amino acid of 3-methyl-2-oxobutanoate hydroxymethyltransferase corresponding to the 159th residue in the amino acid sequence of SEQ ID NO: 37 may be substituted with another amino acid which is selected from the group consisting of arginine (R), histidine (H), lysine (K), aspartic acid (D), glutamic acid (E), serine(S), threonine (T), asparagine (N), glutamine (Q), cysteine (C), proline (P), valine (V), isoleucine (I), leucine (L), methionine (M) and tyrosine (Y), and is different from the original amino acid, and
(2) an amino acid of 3-methyl-2-oxobutanoate hydroxymethyltransferase corresponding to the 116th residue in the amino acid sequence of SEQ ID NO: 37 may be substituted with another amino acid which is selected from the group consisting of aspartic acid (D), glutamic acid (E), serine(S), threonine (T), asparagine (N), glutamine (Q), cysteine (C), alanine (A), valine (V), isoleucine (I), leucine (L) and methionine (M), and is different from the original amino acid.

It is obvious that even if some amino acid sequences except for the amino acid corresponding to the 159th residue and the amino acid corresponding to the 116th residue are deleted, modified, substituted or added in the amino acid sequence of SEQ ID NO: 37, as long as they show the activity of 3-methyl-2-oxobutanoate hydroxymethyltransferase, they may be included in the variant of the present application.

Furthermore, in one example, the variant may comprise a polypeptide in which (1) an amino acid corresponding to the 159th residue of the amino acid sequence of SEQ ID NO: 37 and (2) an amino acid corresponding to the 116th residue of the amino acid sequence of SEQ ID NO: 37 are substituted with another amino acid, in the amino acid sequence having homology or identity of at least 80%, 85%, 90%, 91%, 92%, 93%, 94%, 95%, 96%, 97%, 98%, or 99% or more, to the amino acid sequence disclosed as SEQ ID NO: 37. In other words, a polypeptide which comprises a substitution with another amino acid at (1) a position corresponding to the 159th residue of the amino acid sequence of SEQ ID NO: 37 and (2) a position corresponding to the 116th residue of the amino acid sequence of SEQ ID NO: 37, has sequence homology or identity of at least 80%, 90%, 95%, 96%, 97%, 98%, or 99% or more and less than 100% to the amino acid sequence of SEQ ID NO: 37 and has 3-methyl-2-oxobutanoate hydroxymethyltransferase activity may be comprised in the variant of the present application.

In one embodiment, the 3-methyl-2-oxobutanoate hydroxymethyltransferase variant may comprise the amino acid sequence of SEQ ID NO: 128, but not limited thereto. It is obvious that even if some amino acid sequences except for (1) the amino acid corresponding to the 159th residue and (2) the amino acid corresponding to the 116th residue are deleted, modified, substituted or added in the variant consisting of the amino acid sequence of SEQ ID NO: 128, as long as they show the activity of 3-methyl-2-oxobutanoate hydroxymethyltransferase, they may be included in the variant of the present application. In addition, in one example, the variant the variant may comprise a polypeptide having homology or identity of at least 80%, 85%, 90%, 91%, 92%, 93%, 94%, 95%, 96%, 97%, 98%, or 99% or more to the amino acid sequence of SEQ ID NO: 128 in which (1) an amino acid corresponding to the 159th residue of the amino acid sequence of SEQ ID NO: 37 and (2) an amino acid corresponding to the 116th residue of the amino acid sequence of SEQ ID NO: 37 are fixed. In other words, a polypeptide which comprises a substitution with another amino acid at (1) a position corresponding to the 159th residue of the amino acid sequence of SEQ ID NO: 37 and (2) a position corresponding to the 116th residue of the amino acid sequence of SEQ ID NO: 37, and has sequence homology or identity of at least 80%, 90%, 95%, 96%, 97%, 98%, or 99% or more and less than 100% to the amino acid sequence of SEQ ID NO: 128 and has 3-methyl-2-oxobutanoate hydroxymethyltransferase activity may be included in the variant of the present application.

### Microorganism

The present description provides,
a microorganism producing pantothenic acid or pantoic acid, comprising
(1) one or more selected from the group consisting of a polypeptide in which an amino acid corresponding to the 159th residue from the N-terminus in the amino acid sequence of SEQ ID NO: 37 is substituted with another amino acid, a polynucleotide encoding the polypeptide and a recombinant vector comprising the polynucleotide; or
(2) (1) above, and one or more selected from the group consisting of a polypeptide in which an amino acid corresponding to the 116th residue from the N-terminus in the amino acid sequence of SEQ ID NO: 37 is substituted with another amino acid, a polynucleotide encoding the polypeptide and a recombinant vector comprising the polynucleotide.

In the present description, the term “microorganism with enhanced activity of 3-methyl-2-oxobutanoate hydroxymethyltransferase” may be that a microorganism which does not have pantothenic acid and/or pantoic acid production ability has pantothenic acid and/or pantoic acid production ability, or has higher pantothenic acid and/or pantoic acid production ability than the original pantothenic acid and/or pantoic acid production ability, by being engineered (mutated) to express the aforementioned polypeptide having 3-methyl-2-oxobutanoate hydroxymethyltransferase activity. In the present description, “microorganism” encompasses single-celled bacteria, and may be interchangeably with “cell”. In the present description, the microorganism before being mutated to express the polypeptide having 3-methyl-2-oxobutanoate hydroxymethyltransferase activity, in order to distinguish from the mutated microorganism, may be expressed as “parent microorganism (or parent strain) or host cell”.

In one example, the microorganism may be one or more kinds selected from the group consisting of the genus Corynebacterium microorganism and the genus Escherichia microorganism and the like. The genus Corynebacterium microorganism may include Corynebacterium glutamicum, Corynebacterium ammoniagenes, Brevibacterium lactofermentum, Brevibacterium flavum, Corynebacterium thermoaminogenes, Corynebacterium efficiens, and the like, but not necessarily limited thereto. Much more specifically, the genus Corynebacterium microorganism may be Corynebacterium glutamicum. The genus Escherichia strain may be Escherichia coli.

In the present description, in the microorganism with enhanced activity of 3-methyl-2-oxobutanoate hydroxymethyltransferase, a gene encoding 3-methyl-2-oxobutanoate hydroxymethyltransferase may be introduced.

In the present description, “microorganism with enhanced activity of 3-methyl-2-oxobutanoate hydroxymethyltransferase” may be a microorganism expressing a polypeptide having 3-methyl-2-oxobutanoate hydroxymethyltransferase activity, in which a mutation (engineering) is introduced so that a parent strain expresses a polypeptide having 3-methyl-2-oxobutanoate hydroxymethyltransferase activity. The microorganism may comprise one or more selected from the group consisting of a polypeptide having 3-methyl-2-oxobutanoate hydroxymethyltransferase activity, a polynucleotide encoding the polypeptide and a recombinant vector comprising the polynucleotide. In one example, the mutation to express the polypeptide having 3-methyl-2-oxobutanoate hydroxymethyltransferase activity may be performed by introducing the aforementioned polynucleotide encoding the polypeptide having 3-methyl-2-oxobutanoate hydroxymethyltransferase activity or recombinant vector comprising the same into a parent strain. The polynucleotide encoding the polypeptide having 3-methyl-2-oxobutanoate hydroxymethyltransferase activity, which is introduced to the parent strain as described above may replace the endogenous gene encoding 3-methyl-2-oxobutanoate hydroxymethyltransferase in the parent strain or be further comprised in addition.

In one embodiment, the microorganism expressing the polypeptide having 3-methyl-2-oxobutanoate hydroxymethyltransferase activity may be Accession No. KCCM12973P (named Corynebacterium glutamicum CV03-5002).

In the present description, that a polynucleotide (may be interchangeably used with “gene”) or a polypeptide (may be interchangeably used with “protein”) “comprises a specific nucleic acid sequence or amino acid sequence, or consists of a specific nucleic acid sequence or amino acid sequence, or is expressed as a specific nucleic acid sequence or amino acid sequence” may mean that the polynucleotide or polypeptide necessarily comprises the specific nucleic acid sequence or amino acid sequence, and it may be interpreted as comprising a “substantially equivalent sequence” in which a mutation (deletion, substitution, modification and/or addition) is added to the specific nucleic acid sequence or amino acid sequence within a range of maintaining the original function and/or desired function of the polynucleotide or polypeptide.

In one example, the nucleic acid sequence or amino acid sequence provided in the present description may include those modified by common mutagenesis methods, for example, direct evolution and/or site-directed mutagenesis, and the like, within a range of maintaining its original function or desired function. In one example, that a polynucleotide or polypeptide “comprises a specific nucleic acid sequence or amino acid sequence, or consists of a specific nucleic acid sequence or amino acid sequence” may mean necessarily comprising (i) the specific nucleic acid sequence or amino acid sequence, or (ii) consisting of or necessarily comprising an amino acid sequence having homology of 60% or more, 70% or more, 80% or more, 85% or more, 90% or more, 91% or more, 92% or more, 93% or more, 94% or more, 95% or more, 96% or more, 97% or more, 98% or more, 99% or more, 99.5% or more, or 99.9% or more to the specific nucleic acid sequence or amino acid sequence within a range of maintaining the original function and/or desired function. In the present description, the desired function may refer to a function of increasing or giving pantothenic acid and/or pantoic acid production ability of a microorganism.

In the nucleic acid sequence disclosed in the present description, various modifications may be made o the coding region, within a range that does not change the amino acid sequence and/or function of a protein expressed from the coding region, in consideration of a codon preferred in a microorganism to express the protein (lysine excretion protein) due to degeneracy of the codon.

In the present description, the term “homology (identity)” refers to a degree of correspondence with a given nucleic acid sequence or amino acid sequence and may be expressed as a percentage (%). In case of the homology to the nucleic acid sequence, it may be determined using for example, algorithm BLAST by documents (See: Karlin and Altschul, Pro. Natl. Acad. Sci. USA, 90, 5873, 1993) or FASTA by Pearson (See: Methods Enzymol., 183, 63, 1990). Based on this algorithm BLAST, programs called BLASTN or BLASTX have been developed (See: http://www.ncbi.nlm.nih.gov).

In one example, the polynucleotide comprising the specific nucleic acid sequence provide in the present description may be interpreted as including polynucleotide fragments comprising the specific nucleic acid sequence or substantially equivalent nucleic acid sequence thereto, as well as a complementary nucleic acid sequence to the specific nucleic acid sequence. Specifically, the polynucleotide having complementarity may be hybridized at a Tm value which can be appropriately controlled by those skilled in the art, if necessary, for example, a Tm value of 55° C., 60° C., 63° C. or 65° C., and may be analyzed under the condition described below: this condition is specifically disclosed in known documents. For example, a condition in which genes having high complementarity of 60% or more, 70% or more, 80% or more, 85% or more, 90% or more, 91% or more, 92% or more, 93% or more, 94% or more, 95% or more, 96% or more, 97% or more, 98% or more, 98% or more, 99.5% or more, or 99.9% or more are hybridized, and genes having complementarity lower than that are not hybridized, or a common southern hybridization washing condition, which is a condition of washing once, specifically, twice or three times, at a salt concentration and temperature, corresponding to 60° C., 1×SSC (saline-sodium citrate buffer), and 0.1% (w/v) SDS (Sodium Dodecyl Sulfate); 60° C., 0.1×SSC, and 0.1% SDS; or 68° C., 0.1×SSC, and 0.1% SDS, and the like may be listed, but not limited thereto. Hybridization requires that two nucleotides n have complementary sequences, or mismatches between bases may be allowed depending on the stringency of hybridization. The term “complementary” may be used to describe a relationship between nucleotide bases capable of hybridizing to each other. For example, in case of DNA, adenosine is complementary to thymine and cytosine is complementary to guanine. The appropriate stringency for hybridizing polynucleotides depends on the length and degree of complementarity of the polynucleotides, which are well known in the art (See Sambrook et al., supra, 9.50-9.51, 11.7-11.8).

The introduction of the polynucleotide or vector may be performed by selecting a known transformation method by those skilled in the art. In the present description, the term “transformation” is a process of introducing a specific polynucleotide or a vector comprising the same into a host cell, and the transformed polynucleotide may be located as inserted into a chromosome or located extrachromosomally in a host cell. As one example, transformation may make a protein encoded by the polynucleotide expressed in a host cell by introducing a polynucleotide encoding a target protein (foreign protein) or a vector comprising the same. In addition, the polynucleotide may comprise DNA and/or RNA encoding a target protein. As long as the polynucleotide may be introduced and expressed into a host cell, the form to be introduced is not limited. For example, the polynucleotide may be introduced into a host cell in the form of an expression cassette, which is a gene construct comprising all elements necessary for self-expression. The expression cassette may comprise expression control elements such as a promoter, a transcription termination signal, a ribosome binding site and/or a translation termination signal, which are operably linked to the polynucleotide in general. The expression cassette may be in the form of an expression vector capable of self-replication. In addition, the polynucleotide may be introduced into a host cell in its own form and operably linked to a sequence required for expression in the host cell. As described above, the term “operably linked” may mean that expression control elements (e.g., promoter) and a polynucleotide are functionally linked so that expression control elements can perform transcription control (e.g., transcription initiation) of a polynucleotide encoding a target protein (foreign protein). Operable linking may be performed using a gene recombination technique known in the art, and for example, it may be performed by common site-specific DNA cleavage and ligation, but not limited thereto.

The method for transforming the polynucleotide into a host cell may be performed by any method for introducing nucleic acid into a cell (microorganism), and it may be performed by appropriately selecting a transformation technique known in the art depending on the host cell. As the known transformation method, electroporation, calcium phosphate (CaPO₄) precipitation, calcium chloride (CaCl₂) precipitation, microinjection, polyethylene glycol (PEG) precipitation (polyethylene glycol-mediated uptake), DEAE-dextran method, cation liposome method, lipofection, lithium acetate-DMSO method, and the like may be exemplified, but not limited thereto.

The introduction (insertion) of the polynucleotide into host cell genome (chromosome) may be performed by appropriately selecting a known method by those skilled in the art, and for example, it may be performed using RNA-guided endonuclease system (or CRISPR system; for example, one or more selected from the group consisting of (a) RNA-guided endonuclease (e.g., Cas9 protein, etc.), encoding gene thereof, or a vector comprising the gene; and (b) guided RNA (e.g., single guide RNA (sgRNA), etc.), encoding DNA thereof, or a mixture comprising the vector comprising DNA (for example, mixture of RNA-guided endonuclease protein and guide RNA, etc.), a complex (for example, ribonucleic acid fusion protein (RNP), a recombinant vector (for example, a vector comprising a RNA-guided endonuclease-encoding gene and guide RNA-encoding DNA together, etc.), and the like), but not limited thereto.

In the present description, the term “vector” means a DNA product containing the nucleotide sequence of the polynucleotide encoding the target protein operably linked to an appropriate regulatory sequence so that a target protein can be expressed in a suitable host. The regulatory sequence may include a promoter capable of initiating transcription, any operator sequence for regulating transcription, a sequence encoding a suitable mRNA ribosome binding site, and/or a sequence for regulating termination of transcription and/or translation. After the vector is transformed into a host cell, it may be expressed independently of the genome of the host cell, or may be integrated into the genome of the host cell.

The vector available in the present description is not particularly limited as long as it is capable of replication in a host cell, and may be selected from all commonly used vectors. Examples of commonly used vectors may include natural or recombinant plasmids, cosmids, viruses, bacteriophages and the like. For example, as the vector, pWE15, M13, MBL3, MBL4, IXII, ASHII, APII, t10, t11, Charon4A, and Charon21A and the like may be used as a phage vector or cosmid vector, and pBR-based, pUC-based, pBluescript II-based, pGEM-based, pTZ-based, pCL-based and pET-based and the like may be used as a plasmid vector, but not limited thereto. Specifically, pDZ, pACYC177, pACYC184, pCL, pECCG117, pUC19, pBR322, pMW118, pCC1BAC vectors, and the like may be exemplified, but not limited thereto.

The vector available in the present description may be a known expression vector and/or a vector for insertion into host cell chromosome of a polynucleotide. The insertion of the polynucleotide into the host cell chromosome may be performed by any method known in the art, for example, homologous recombination or CRISPR system, but not limited thereto. The vector may further comprise a selection marker for confirming whether or not it is inserted into the chromosome. The selection marker is to select cells transformed into a vector, that is, to confirm whether the polynucleotide is inserted, and it may be selected and used among genes conferring a selectable phenotype, such as drug resistance, auxotrophic requirement, resistance to cytotoxic agents, or expression of surface protein. In an environment treated with a selective agent, only cells expressing a selectable marker survive or exhibit other phenotypic characteristics, and thus transformed cells may be selected.

Other example, provides a composition for producing pantothenic acid or pantoic acid, comprising the microorganism.

Other example provides a method for preparation of pantothenic acid or pantoic acid, comprising culturing the microorganism in a medium.

Other example provides a method for increasing pantothenic acid and/or pantoic acid production ability of the microorganism or a method for giving pantothenic acid production ability to the microorganism, comprising enhancing 3-methyl-2-oxobutanoate hydroxymethyltransferase activity of a microorganism.

The enhancing activity of 3-methyl-2-oxobutanoate hydroxymethyltransferase of the microorganism may comprise introducing a mutation so as to express a polypeptide having 3-methyl-2-oxobutanoate hydroxymethyltransferase activity into the microorganism.

The introducing a mutation may comprise introducing (transforming) an encoding polynucleotide of a polypeptide having 3-methyl-2-oxobutanoate hydroxymethyltransferase activity or a recombinant vector comprising the polynucleotide into a microorganism.

Other example provides a method for production of pantothenic acid and/or pantoic acid, comprising culturing the aforementioned microorganism with enhanced activity of 3-methyl-2-oxobutanoate hydroxymethyltransferase in a medium. The method, may further comprise recovering pantothenic acid and/or pantoic acid from the cultured microorganism, medium or both of them, after the culturing.

In the method, the culturing a microorganism is not particularly limited thereto, but may be performed by known batch culturing methods, continuous culturing methods, fed-batch culturing methods, and the like. Then, the culturing condition, is not particularly limited thereto, but may adjust an appropriate pH (For example, pH 5 to 9, specifically, pH 6 to 8, most specifically, pH 6.8) using a basic compound (e.g.: sodium hydroxide, potassium hydroxide or ammonia) or acidic compound (e.g.: phosphate or sulfate), and may maintain an aerobic condition by introducing oxygen or an oxygen-containing gas mixture to the culture. The culturing temperature may be maintained by 20 to 45° C., or 25 to 40° C., and the culturing may be conducted for about 10 to 160 hours, but not limited thereto. The pantothenic acid and/or pantoic acid produced by the culturing may be secreted to the medium or remain in cells.

The medium available for the culturing may use one or more kinds selected from the group consisting of sugar and carbohydrate (e.g.: glucose, sucrose, lactose, fructose, maltose, molasse, starch and cellulose), oil and fat (e.g.: soybean oil, sunflower seed oil, peanut oil and coconut oil), fatty acid (e.g.: palmitic acid, stearic acid and linoleic acid), alcohol (e.g.: glycerol and ethanol), organic acid (e.g.: acetic acid), and the like individually or use them by mixing two or more kinds as a carbon source, but not limited thereto. As a nitrogen source, one or more kinds selected from the group consisting of nitrogen-containing organic compound (e.g.: peptone, yeast extract, meat juice, malt extract, corn steep liquid, soybean meal powder and urea), inorganic compound (e.g.: ammonium sulfate, ammonium chloride, ammonium phosphate, ammonium carbonate and ammonium nitrate), and the like may be used individually, or two or more kinds may be mixed and used, but not limited thereto. As a phosphorus source, one or more kinds selected from the group consisting of potassium dihydrogen phosphate, dipotassium hydrogen phosphate, sodium-containing salts corresponding thereto, and the like may be used individually, or two or more kinds may be mixed and used, but not limited thereto. In addition, the medium may comprise essential growth-promoting substances such as other metal salts (e.g.: magnesium sulfate or iron sulfate), amino acids and/or vitamins, and the like.

The recovering pantothenic acid and/or pantoic acid may collecting desired amino acids from a medium, culture solution or microorganism, using a known appropriate method in the art depending on the culturing method. For example, the recovering may be performed by one or more methods selected from centrifugation, filtration, anion exchange chromatography, crystallization, HPLC, and the like. The method for recovering pantothenic acid and/or pantoic acid may additionally comprise a purification step before that, at the same time, or after that.

### Advantageous Effects

In the present description, 3-methyl-2-ketobutanoate hydroxymethyltransferase variant is provided, and a technology for increasing the production ability of pantothenic acid and/or pantoic acid of a microorganism using the same is provided. By introducing a mutation to express the 3-methyl-2-ketobutanoate hydroxymethyltransferase variant into a microorganism, a technology capable of improving the productivity of pantothenic acid and/or pantoic acid or giving the productivity of pantothenic acid and/or pantoic acid is provided.

### MODE FOR INVENTION

Hereinafter, the present invention will be more specifically described by examples, but they are illustrative only, and are not intended to limit the scope of the present invention. It is obvious to those skilled in the art that the examples described below may be modified without departing from the essential gist of the invention.

### Example 1. 3-Methyl-2-oxobutanoate Hydroxymethyltransferase Gene Search and Selection

As the result of NCBI BLAST search by setting a gene encoding 3-methyl-2-oxobutanoate hydroxymethyltransferase (panB) of Corynebacterium glutamicum ATCC13032 to a query, candidate genes estimated as having activity of the gene encoding 3-methyl-2-oxobutanoate hydroxymethyltransferase and microorganisms having them were selected. Among them, the genes encoding 3-methyl-2-oxobutanoate hydroxymethyltransferase derived from microorganisms having a biosafety level of 1 were selected, and they are summarized in Table 1:

TABLE 1
Primers/plasmids used for selection of microorganisms estimated to
have a gene encoding 3-methyl-2-oxobutanoate hydroxymethyltransferase
and 3-methyl-2-oxobutanoate hydroxymethyltransferase genes

| | | KCTC | | |
|---|---|---|---|---|
| | Microorganism | Accession | | |
| | name | No. | Primer | Plasmid |
| 1 | Escherichia coli | ATCC47076 | SEQ ID NOs: 1, | pECCG117- |
| | | | 2 | panB(EC) |
| 2 | Bacillus | KCTC3135(ATCC 6051) | SEQ ID NOs: 3, | pECCG117- |
| | subtilis | | 4 | panB(BS) |
| 3 | Pantoea | KCTC2564(ATCC27155) | SEQ ID NOs: 5, | pECCG117- |
| | agglomerans | | 6 | panB(PA) |
| 4 | Serratia | KCTC2927(ATCC27593) | SEQ ID NOs: 7, | pECCG117- |
| | rubidaea | | 8 | panB(SR) |
| 5 | Serratia | KCTC2936(ATCC19323) | SEQ ID NOs: 9, | pECCG117- |
| | proteamaculans | | 10 | panB(SP) |
| 6 | Pseudomonas | KCTC12498(ATCC14235) | SEQ ID NOs: | pECCG117- |
| | resinovorans | | 11, 12 | panB(PR) |
| 7 | Pedobacter | KCTC12762(DSM17933) | SEQ ID NOs: | pECCG117- |
| | terrae | | 13, 14 | panB(PT) |
| 8 | Citrobacter | KCTC42139(JCM30009) | SEQ ID NOs: | pECCG117- |
| | bitternis | | 15, 16 | panB(CB) |
| 9 | Enterobacter | KCTC2519(ATCC23355) | SEQ ID NOs: | pECCG117- |
| | cloacae | | 17, 18 | panB(ECl) |
| 10 | Achromobacter | KCTC22890(ATCC43552) | SEQ ID NOs: | pECCG117- |
| | piechaudii | | 19, 20 | panB(AP) |
| 11 | Staphylococcus | KCTC1917(ATCC12228) | SEQ ID NOs: | pECCG117- |
| | epidermidis | | 21, 22 | panB(SE) |
| 12 | Shigella | KCTC12073 | SEQ ID NOs: | pECCG117- |
| | flexneri | | 23, 24 | panB(SF) |
| 13 | Corynebacterium | KCTC9097(ATCC13032) | SEQ ID NOs: | pECCG117- |
| | glutamicum | | 25, 26 | panB(CG) |

### Example 2. Production of the Genus Corynebacterium Microorganism in which Foreign Microorganism-Derived 3-Methyl-2-Oxobutanoate Hydroxymethyltransferase

After extracting genome of the microorganisms secured in Example 1, PCR was performed using the primer sequences of Table 1 using it as a template, and DNA fragments encoding 3-methyl-2-oxobutanoate hydroxymethyltransferase were amplified. The PCR was performed using PfuUltra™ high-confidence DNA polymerase (Stratagene), and was performed under the condition of repeating denaturation at 95° C. for 30 seconds; annealing at 55° C. for 30 seconds; and polymerization at 72° C. for 1 minute 30 times. As a result, DNA fragments encoding each 3-methyl-2-oxobutanoate hydroxymethyltransferase (panB) were obtained.

In order to secure Corynebacterium glutamicum-derived PLM1 promoter, PCR was performed using primers of SEQ ID NOs: 27 and 28 using Corynebacterium glutamicum (ATCC13032) genome as a template as same as described above to obtain promoter DNA fragments.

After treating with restriction enzyme BamHI, by cloning pECCG117 vector heat-treated at 65° C. for 20 minutes (Korean Patent No. 10-0057684) and obtained DNA fragments (each panB, PLM1 promoter) to be a molarity (M) of 2:1:1 (pECCG117 vector: panB: PLM1) according to the provided manual using Infusion Cloning Kit of TakaRa, plasmids were obtained, and the names of the obtained plasmids and introduced gene information were marked in Table 1 above.

By transforming the produced 13 kinds of vectors into Corynebacterium glutamicum ATCC13032 by electroporation, strains expressing foreign PanB (3-methyl-2-oxobutanoate hydroxymethyltransferase) were produced.

### Example 3. Investigation of Pantothenic Acid Production Ability of the Genus Corynebacterium Microorganism in which Foreign Microorganism-Derived 3-Methyl-2-Oxobutanoate Hydroxymethyltransferase is Expressed

In order to confirm the productivity of pantothenic acid of various foreign microorganism-derived panB expressing strains obtained in Example 2, a parent strain (non-transformed strain) and the strains were inoculated into a 250 ml corner-baffle flask containing production medium 25 ml consisting of the following composition, respectively, and then cultured with shaking at 200 rpm at 32° C. for 48 hours to prepare pantothenic acid.

### <Production Medium>

Glucose 10%, beta-alanine 0.5%, yeast extract 0.4%, ammonium sulfate 1.5%, potassium phosphate monobasic 0.1%, magnesium sulfate 7 hydrate 0.05%, iron sulfate 7 hydrate 10 mg/l, manganese sulfate 1 hydrate 6.7 mg/l, biotin 50 μg/l, thiamine·HCl 100 μg/l, pH 7.2

The obtained culture solution was centrifuged at 20,000 rcf for 10 minutes, and then the supernatant was diluted by 1/10 with TDW (triple distilled water), and then HPLC analysis was performed to measure the concentration of pantothenic acid and L-valine, and the result was shown in Table 2 below.

TABLE 2

| | Pantothenic | |
|---|---|---|
| | acid | L-valine |
| | concentration | concentration |
| | (g/L) | (g/L) |
| ATCC13032 (wild type) | 0.0 | 2.4 |
| ATCC13032 pECCG117-panB(EC) | 1.2 | 1.5 |
| ATCC13032 pECCG117-panB(BS) | 0.5 | 1.9 |
| ATCC13032 pECCG117-panB(PA) | 0.6 | 2.0 |
| ATCC13032 pECCG117-panB(SR) | 0.7 | 1.8 |
| ATCC13032 pECCG117-panB(SP) | 0.3 | 2.2 |
| ATCC13032 pECCG117-panB(PR) | 0.7 | 1.9 |
| ATCC13032 pECCG117-panB(PT) | 0.7 | 2.0 |
| ATCC13032 pECCG117-panB(CB) | 0.7 | 2.0 |
| ATCC13032 pECCG117-panB(ECl) | 0.6 | 2.1 |
| ATCC13032 pECCG117-panB(AP) | 0.6 | 1.9 |
| ATCC13032 pECCG117-panB(SE) | 0.5 | 2.0 |
| ATCC13032 pECCG117-panB(SF) | 0.7 | 1.9 |
| ATCC13032 pECCG117-panB(CG) | 0.6 | 2.0 |

As shown in Table 2, the parent strain, Corynebacterium glutamicum ATCC 13032 did not produce pantothenic acid, whereas all the tested Corynebacterium glutamicum strains expressing foreign microorganism-derived panB produced about 0.6 g/L pantothenic acid. In particular, among the foreign microorganism-derived PanB expressing strains, E. coli-derived panB expressing strain, ATCC13032 pECCG117-panB (EC) showed the highest pantothenic acid productivity (1.2 g/L).

The above result showed that 13 kinds of microorganism-derived enzymes (3-methyl-2-oxobutanoate hydroxymethyltransferase) selected in Example 1 showed the pantothenic acid production ability, and among them, the E. coli-derived enzyme showed especially high pantothenic acid production ability.

### Example 4. Production of the Genus Corynebacterium Microorganism in which E. Coli-Derived 3-Methyl-2-Oxobutanoate Hydroxymethyltransferase Gene is Introduced

A plasmid to introduce the E. coli-derived 3-methyl-2-oxobutanoate hydroxymethyltransferase-encoding gene (panB) determined as having the especially excellent pantothenic acid production ability in Example 3 into Corynebacterium glutamicum ATCC13032 was produced.

At first, a vector to delete panB present in the parent strain was produced. PCR was performed using primers of SEQ ID NOs: 29 and 30 and SEQ ID NOs: 31 and 32 using the genome DNA of Corynebacterium glutamicum ATCC13032 as a template. PCR was performed under the condition of repeating denaturation at 95° C. for 30 seconds; annealing at 55° C. for 30 seconds; and polymerization at 72° C. for 1 minute 25 times. As a result, gene fragments of 1000 bp at the top region of the panB gene and 1000 bp at the bottom region of the panB gene were obtained, respectively, and each amplified product was purified using PCR Purification kit of QIAGEN and was used as insertion DNA fragments for vector production.

After treating with restriction enzyme smaI, by cloning pDZ vector heat-treated at 65° C. for 20 minutes (Korean Patent No. 0924065) and DNA fragments (gene fragments of 1000 bp at the top region of panB gene and gene fragments of 1000 bp at the bottom region of panB gene) to be a molarity (M) of 2:1:1 according to the provided manual using Infusion Cloning Kit of TakaRa, a vector to delete panB gene on chromosome, pDZ_ΔpanB was produced.

In order to prepare E. coli-derived panB gene, PCR was performed using primers of SEQ ID NOs: 33 and 34 using the plasmid pECCG117-panB (EC) produced in Example 2 as a template. PCR was performed by repeating denaturation at 95° C. for 30 seconds; annealing at 55° C. for 30 seconds; and polymerization at 72° C. for 1 minute 25 times, and as a result, DNA fragments of 1077 bp were obtained. After treating with restriction enzyme smaI, by cloning pDZ_ΔpanB vector heat-treated at 65° C. for 20 minutes and the obtained DNA fragments to be a molarity (M) of 1:2 according to the provided manual using Infusion Cloning Kit of TakaRa, a vector to introduce E. coli-derived panB gene on chromosome, pDZ_ΔpanB::PLM1-panB (EC) was produced.

The produced vectors, pDZ_ΔpanB and pDZ_ΔpanB::panB (EC) were transformed into Corynebacterium glutamicum ATCC13032, respectively, by electroporation, and through a secondary cross process, a strain in which panB was deleted (ΔpanB strain) and a strain in which E. coli-derived panB was introduced (ΔpanB::panB (EC)) were obtained on chromosome, respectively. Whether or not the appropriate substitution of E. coli-derived panB was confirmed using the following primer combination using MASA (Mutant Allele Specific Amplification) PCR technique (Takeda et al., Hum. Mutation, 2, 112-117 (1993)). In other words, primary determination was made by selecting strains amplified in the primer combination corresponding to E. coli panB (SEQ ID NOs: 35 and 28 and SEQ ID NOs: 36 and 1), and secondary confirmation was conducted by analyzing the panB sequence of the selected strain using the primer combination of SEQ ID NO: 35 and SEQ ID NO: 36.

In order to confirm the pantothenic acid productivity of the mutant strains obtained as described above, after inoculating Corynebacterium glutamicum ATCC 13032 wild type strain, ΔpanB strain, and ΔpanB::panB (EC) mutant strain into a 250 ml corner-baffle flask containing production medium (see Example 3) 25 ml, they were cultured with shaking at 300 rpm at 32° C. for 48 hours to prepare pantothenic acid.

The obtained culture solution was centrifuged at 20,000 rcf for 10 minutes, and then the supernatant was diluted by 1/10 with TDW (triple distilled water), and then HPLC analysis was performed to measure the concentration of pantothenic acid and L-valine, and the result was shown in Table 3 below.

TABLE 3

| | | L-valine |
|---|---|---|
| | Pantothenic acid | concentration |
| | concentration (g/L) | (g/L) |
| ATCC13032 (wild type) | 0.1 | 1.9 |
| ATCC13032 ΔpanB | 0.0 | 2.7 |
| ATCC13032 | 0.4 | 1.3 |
| ΔpanB::panB(EC) | | |

As shown in Table 3, the Corynebacterium glutamicum ATCC 13032 wild type and panB deleted (ΔpanB) strain did not produce pantothenic acid at all or hardly produced it, whereas the mutant Corynebacterium glutamicum expressing foreign panB (ΔpanB::panB (EC)) produced pantothenic acid at a concentration of 0.4g/l.

### Example 5. Random Mutant Strain Production Through Artificial Mutation (NTG-Based Mutation) and panB Producing Strain Selection

In the present example, in order to obtain a microorganism mutant strain with much more enhanced production ability of pantothenic acid, using the following method, a mutation of the microorganism was induced using the mutant Corynebacterium glutamicum ATCC13032 ΔpanB::panB (EC) strain expressing E. coli-derived panB produced according to Example 4 as a parent strain.

Specifically, the Corynebacterium glutamicum ATCC13032 ΔpanB::panB (EC) strain was activated by culturing it in an activation medium for 16 hours, and was inoculated in a seed medium sterilized at 121° C. for 15 minutes and cultured for 14 hours, and then the culture solution 5 ml was recovered. After washing the recovered culture solution with 100 mM citric buffer, NTG (N-Methyl-N′-nitro-N-nitrosoguanidine) was added so as to be the final concentration of 200 mg/l and was treated for 20 minutes, and was washed with 100 mM phosphate buffer. As the result of measuring the death rate by smearing the NTG-treated strain to a minimal medium, the death rate was shown as 85%. The survived cells were inoculated and cultured, and finally, 2 kinds of mutant strains showing the excellent pantothenic acid production ability were selected and named Corynebacterium glutamicum CJVB5-01 and CJVB5-02 (Corynebacterium glutamicum, CJVB5-02).

The composition of the medium used in the present example was as follows.

### <Activation Medium>

Beef extract 1%, polypeptone 1%, sodium chloride 0.5%, yeast extract 1%, agar 2%, pH 7.2

### <Production Medium>

Glucose 10%, beta-alanine 0.5%, yeast extract 0.4%, ammonium sulfate 1.5%, potassium phosphate monobasic 0.1%, magnesium sulfate 7 hydrate 0.05%, iron sulfate 7 hydrate 10 mg/l, manganese sulfate 1 hydrate 6.7 mg/l, biotin 50 μg/l, thiamine·HCl 100 g/l, pH 7.2

### <Minimal Medium>

Glucose 1.0%, ammonium sulfate 0.4%, magnesium sulfate 0.04%, potassium phosphate monobasic 0.1%, urea 0.1%, thiamine 0.001%, biotin 200 μg/l, agar 2%, pH 7.2

In order to confirm the pantothenic acid production ability of the obtained mutant strains Corynebacterium glutamicum CJVB5-01 and CJVB5-02, after inoculating Corynebacterium glutamicum ATCC13032 ΔpanB::panB (EC) strain, CJVB5-01 mutant strain and CJVB5-02 mutant strain into a 250 ml corner-baffle flask containing production medium 25 ml, respectively, they were cultured with shaking at 200 rpm at 32° C. for 48 hours to prepare pantothenic acid.

The obtained culture solution was centrifuged at 20,000 rcf for 10 minutes, and then the supernatant was diluted by 1/10 with TDW (triple distilled water), and then HPLC analysis was performed to measure the concentration of pantothenic acid and L-valine, and the result was shown in Table 4 and Table 5 below.

TABLE 4

| | Pantothenic acid | L-valine |
|---|---|---|
| | concentration | concentration |
| | (g/L) | (g/L) |
| ATCC13032 ΔpanB | 0.0 | 2.4 |
| ATCC13032 | 0.3 | 1.4 |
| ΔpanB::panB(EC) | | |
| CJVB5-01 | 1.2 | 1.0 |

TABLE 5

| | Pantothenic acid | L-valine |
|---|---|---|
| | concentration (g/L) | concentration (g/L) |
| ATCC13032 | 0.3 | 0.9 |
| ΔpanB::panB(EC) | | |
| CJVB5-02 | 1.7 | 0.2 |

As shown in Table 4 and Table 5 above, it was confirmed that Corynebacterium glutamicum ΔpanB strain did not produce pantothenic acid, and the Corynebacterium glutamicum CJVB5-01 mutant strain and CJVB5-02 mutant strain showed more excellent pantothenic acid production ability, compared to the foreign panB-inserted Corynebacterium glutamicum ΔpanB::panB (EC). In addition, it could be confirmed that the pantothenic acid production ability of the CJVB5-1 mutant strain and CJVB5-02 mutant strain was much stronger than that of the wild type, considering that the concentration of valine, a substance using 3-methyl-2-oxobutanoate as a substrate decreased.

As the result of genome sequencing of the Corynebacterium glutamicum CJVB5-01 mutant strain, it was confirmed that the inserted E. coli panB gene was mutated to encode a variant in which G116A mutation (substitution of the 116th amino acid residue in the amino acid sequence of SEQ ID NO: 37, G (Gly) with A (Ala)) was introduced into wild type E. coli 3-methyl-2-oxobutanoate hydroxymethyltransferase (SEQ ID NO: 37). Hereinafter, the indication of amino acid mutations using amino acid positions such as ‘G116A’ is understood to mean amino acid mutations and/or genetic mutations inducing such amino acid mutations. The amino acid sequence of of the E. coli 3-methyl-2-oxobutanoate hydroxymethyltransferase variant in which the G116A mutation was introduced was shown in SEQ ID NO: 62.

As the result of genome sequencing of the Corynebacterium glutamicum CJVB5-02 mutant strain, it was confirmed that the inserted E. coli panB gene was mutated to encode a variant in which A159L mutation (substitution of the 159th amino acid residue in the amino acid sequence of SEQ ID NO: 37, A (Ala, alanine) with L (Leu, leucine)) was introduced into wild type E. coli 3-methyl-2-oxobutanoate hydroxymethyltransferase (SEQ ID NO: 37). Hereinafter, the indication of amino acid mutations using amino acid positions such as ‘A159L’ is understood to mean amino acid mutations and/or genetic mutations inducing such amino acid mutations. The amino acid sequence of the E. coli 3-methyl-2-oxobutanoate hydroxymethyltransferase variant in which the A159L mutation was introduced was shown in SEQ ID NO: 110.

As a result, it was confirmed that the CJVB5-01 mutant strain and CJVB5-02 mutant strain produced pantothenic acid with high efficiency and high yield, without inhibiting a pathway of synthesizing pantothenic acid from pyruvic acid.

### Example 6. Production of Mutant panB Plasmid Having 3-Methyl-2Ketobutanoate Hydroxymethyltransferase Activity

In order to confirm that the amino acid residues corresponding to the 116th and/or 159th residues, positions of mutations of E. coli PanB (3-methyl-2-oxobutanoate hydroxymethyltransferase) confirmed as affecting the pantothenic acid production ability through Example 5 were important positions, a mutant in which amino acid residues at these positions were substituted with other amino acids was produced and the effect was confirmed.

Using primers described in Table 6 below using pECCG117-panB (EC) produced in Example 2 (See Table 1) as a template, a variant in which random mutagenesis (saturated mutagenesis) in which the amino acid at the 116th position of E. coli PanB (SEQ ID NO: 37), G (Gly) was substituted with another amino acid was introduced (that is, the panB gene mutated to encode E. coli PanB in which the random mutagenesis was introduced was introduced) was produced. In addition, using primers described in Table 7 below, a variant, in which random mutagenesis in which the amino acid at the 159th position of E. coli PanB (SEQ ID NO: 37), A (Ala) was substituted with another amino acid was introduced, was produced. The pECCG117-panB (EC) produced in Example 1 was used as a template. The amino acids substituted according to the mutant of the mutant strain in which each saturated mutagenesis was introduced and primers used for each mutant were summarized in Table 6 and Table 7 below:

TABLE 6

| | Amino acid | |
|---|---|---|
| Template | substitution | Used primer |
| pECCG117-panB(EC) | G116S | SEQ ID NOs: 27, 38/39, 28 |
| | G116C | SEQ ID NOs: 27, 40/41, 28 |
| | G116L | SEQ ID NOs: 27, 42/43, 28 |
| | G116I | SEQ ID NOs: 27, 44/45, 28 |
| | G116T | SEQ ID NOs: 27, 46/47, 28 |
| | G116V | SEQ ID NOs: 27, 48/49, 28 |
| | G116M | SEQ ID NOs: 27, 50/51, 28 |
| | G116D | SEQ ID NOs: 27, 52/53, 28 |
| | G116E | SEQ ID NOs: 27, 54/55, 28 |
| | G116N | SEQ ID NOs: 27, 56/57, 28 |
| | G116Q | SEQ ID NOs: 27, 58/59, 28 |
| | G116A | SEQ ID NOs: 27, 60/61, 28 |

TABLE 7

| | Substituted amino | |
|---|---|---|
| Template | acid | Used primer |
| pECCG117- | A159R | SEQ ID NOs: 74, 75/76, 77 |
| panB(EC) | A159S | SEQ ID NOs: 74, 78/79, 77 |
| | A159Y | SEQ ID NOs: 74, 80/81, 77 |
| | A159C | SEQ ID NOs: 74, 82/83, 77 |
| | A159P | SEQ ID NOs: 74, 84/85, 77 |
| | A159H | SEQ ID NOs: 74, 86/87, 77 |
| | A159L | SEQ ID NOs: 74, 88/89, 77 |
| | A159I | SEQ ID NOs: 74, 90/91, 77 |
| | A159T | SEQ ID NOs: 74, 92/93, 77 |
| | A159K | SEQ ID NOs: 74, 94/95, 77 |
| | A159V | SEQ ID NOs: 74, 96/97, 77 |
| | A159M | SEQ ID NOs: 74, 98/99, 77 |
| | A159D | SEQ ID NOs: 74, 100/101, 77 |
| | A159E | SEQ ID NOs: 74, 102/103, 77 |
| | A159N | SEQ ID NOs: 74, 104/105, 77 |
| | A159Q | SEQ ID NOs: 74, 106/107, 77 |
| | WT (SEQ ID NO: 37) | SEQ ID NOs: 74, 77 |

Specifically, using primers presented in Table 6 and Table 7 above, PCR was performed using pECCG117-panB (EC) (See Table 1) produced in Example 2 as a template. As polymerase, Solg™ Pfu-X DNA polymerase (SolGent co., Ltd.) was used, and PCR was progressed by denaturing at 95° C. for 10 minutes, and then repeating denaturation at 95° C. for 30 seconds, annealing at 55° C. for 30 seconds and polymerization at 72° C. for 1 minute 25 times, and then performing polymerization at 72° C. for 5 minutes. As a result, 610 bp DNA fragments at the 5′ upstream region and 470 bp DNA fragments at the 3′ downstream region centering on the mutation (116th residue) of 3-methyl-2-oxobutanoate hydroxymethyltransferase gene were obtained, and 477 bp DNA fragments at the 5′ upstream region and 318 bp DNA fragments at the 3′ downstream region centering on the 159th residue were obtained.

After treating with restriction enzyme BamHI, by cloning pECCG117 vector heat-treated at 65° C. for 20 minutes (Korean Patent No. 10-0057684) and obtained each DNA fragment (116th residue: 610 bp DNA fragments at the 5′ upstream region and 470 bp DNA fragments at the 3′ downstream region, 159th residue: 477 bp DNA fragments at the 5′ upstream region and 318 bp DNA fragments at the 3′ downstream region) to be a molarity (M) of 2:1:1 according to the provided manual using Infusion Cloning Kit of TakaRa, a plasmid for mutant panB introduction was obtained. In order to secure Corynebacterium glutamicum-derived PLM1 promoter, PCR was performed using Corynebacterium glutamicum (ATCC13032) genome DNA as a template using primers of SEQ ID NOs: 108 and 109 as same as described above, to obtain promoter DNA fragments.

The information of the obtained mutant plasmids was summarized in Table 8 and Table 9 below:

TABLE 8

| | | Mutant plasmid produced |
|---|---|---|
| Mutation | Amino acid | to induce amino acid |
| position | substitution | substitution |
| Amino acid | G116S | pECCG117-panB(G116S) |
| residue | G116C | pECCG117-panB(G116C) |
| corresponding | G116L | pECCG117-panB(G116L) |
| to the | G116I | pECCG117-panB(G116I) |
| 116th | G116T | pECCG117-panB(G116T) |
| residue of | G116V | pECCG117-panB(G116V) |
| Wild type | G116M | pECCG117-panB(G116M) |
| panB (SEQ | G116D | pECCG117-panB(G116D) |
| ID NO: 37) | G116E | pECCG117-panB(G116E) |
| | G116N | pECCG117-panB(G116N) |
| | G116Q | pECCG117-panB(G116Q) |
| | G116A | pECCG117-panB(G116A) |

TABLE 9

| | | Mutant plasmid produced |
|---|---|---|
| Mutation | Amino acid | to induce amino acid |
| position | substitution | substitution |
| Amino acid | A159R | pECCG117-panB(A159R) |
| residue | A159S | pECCG117-panB(A159S) |
| corresponding | A159Y | pECCG117-panB(A159Y) |
| to the | A159C | pECCG117-panB(A159C) |
| 116th | A159P | pECCG117-panB(A159P) |
| residue of | A159H | pECCG117-panB(A159H) |
| Wild type | A159L | pECCG117-panB(A159L) |
| panB (SEQ | A159I | pECCG117-panB(A159I) |
| ID NO: 37) | A159T | pECCG117-panB(A159T) |
| | A159K | pECCG117-panB(A159K) |
| | A159V | pECCG117-panB(A159V) |
| | A159M | pECCG117-panB(A159M) |
| | A159D | pECCG117-panB(A159D) |
| | A159E | pECCG117-panB(A159E) |
| | A159N | pECCG117-panB(A159N) |
| | A159Q | pECCG117-panB(A159Q) |
| | WT | pECCG117-panB(WT) |

### Example 7. Evaluation of Pantothenic Acid Production Ability of 3-Methyl-2Ketobutanoate Hydroxymethyltransferase

The mutant plasmid produced in Example 6 (Table 8 and Table 9) and pECCG117-panB (WT-EC) (Table 1) were introduced into the ATCC13032 ΔpanB strain produced in Example 4 by an electric pulse method, and then they were smeared in a selective medium containing kanamycin 25 mg/l to obtain total 19 kinds of transformed mutant strains in which each random mutagenesis (saturated mutagenesis) was introduced. Then, by progressing flask evaluation by the same method as Example 3, the production ability of the obtained pantothenic acid was measured. The obtained result was shown in Table 10 and Table 11:

TABLE 10

| | Pantothenic acid (g/L) | | | |
|---|---|---|---|---|
| | Arrangement | Arrangement | Arrangement | |
| Strain | 1 | 2 | 3 | Average |
| ATCC 13032 ΔpanB (Control group) | 0.0 | 0.0 | 0.0 | 0.0 |
| ATCC 13032 ΔpanB pECCG117- | 1.5 | 1.9 | 1.6 | 1.7 |
| panB(G116S) | | | | |
| ATCC 13032 ΔpanB pECCG117- | 1.2 | 1.3 | 1.2 | 1.2 |
| panB(G116C) | | | | |
| ATCC 13032 ΔpanB pECCG117- | 1.6 | 1.5 | 1.4 | 1.5 |
| panB(G116L) | | | | |
| ATCC 13032 ΔpanB pECCG117- | 1.5 | 1.7 | 1.6 | 1.6 |
| panB(G116I) | | | | |
| ATCC 13032 ΔpanB pECCG117- | 2.4 | 2.5 | 2.3 | 2.4 |
| panB(G116T) | | | | |
| ATCC 13032 ΔpanB pECCG117- | 1.6 | 1.7 | 1.4 | 1.6 |
| panB(G116V) | | | | |
| ATCC 13032 ΔpanB pECCG117- | 0.9 | 0.9 | 1.1 | 1.0 |
| panB(G116M) | | | | |
| ATCC 13032 ΔpanB pECCG117- | 1.3 | 1.1 | 1.2 | 1.2 |
| panB(G116D) | | | | |
| ATCC 13032 ΔpanB pECCG117- | 1.9 | 1.2 | 2.1 | 1.7 |
| panB(G116E) | | | | |
| ATCC 13032 ΔpanB pECCG117- | 2.5 | 2.5 | 2.6 | 2.5 |
| panB(G116N) | | | | |
| ATCC 13032 ΔpanB pECCG117- | 1.0 | 1.1 | 1.0 | 1.0 |
| panB(G116Q) | | | | |
| ATCC 13032 ΔpanB pECCG117- | 2.6 | 2.9 | 2.9 | 2.8 |
| panB(G116A) | | | | |
| ATCC 13032 ΔpanB pECCG117- | 0.9 | 0.8 | 1.0 | 0.9 |
| panB(WT) | | | | |

As could be seen in Table 10 above, the ATCC13032 ΔpanB strain did not produce pantothenic acid, whereas all the E. coli PanB (wild type) or mutant strains in which its mutant was introduced showed the pantothenic acid production ability. In addition, the mutant strains in which the G116S, G116C, G116L, G116I, G116T, G116V, G116D, G116E, G116N, G116A, G116M, or G116Q mutation was introduced produced pantothenic acid at a much higher level compared to ATCC 13032 ΔpanB pECCG117-panB (WT) which was the mutant strain comprising the E. coli PanB (wild type). As a result, both the wild type of E. coli PanB and mutant had the effect of increasing pantothenic acid production, and in particular, it could be confirmed that the 116th amino acid residue of PanB (SEQ ID NO: 37) was the important position for pantothenic acid production, and when the amino acid at this position was substituted with various amino acids different from the original one, the production ability of pantothenic acid was further increased.

The ATCC 13032 ΔpanB pECCG117-panB (G116A) strain (named Corynebacterium glutamicum CV03-5001) confirmed as having the most excellent production ability of pantothenic acid in the present example was deposited to Korean Culture Center of Microorganisms located in Hongje-dong, Seodaemun-gu, Seoul, Korea on Jun. 8, 2020 and was given an accession number of KCCM12744P.

TABLE 11
Pantothenic acid production ability of saturated mutagenesis-introduced strain

| | Pantothenic acid (g/L) | | | |
|---|---|---|---|---|
| | Arrangement | Arrangement | Arrangement | |
| Strain | 1 | 2 | 3 | Average |
| ATCC 13032 | 0.0 | 0.0 | 0.0 | 0 |
| ATCC 13032 pECCG117- | 0.7 | 0.8 | 0.8 | 0.77 |
| panB(A159R) | | | | |
| ATCC 13032 pECCG117- | 2.5 | 2.9 | 2.6 | 2.67 |
| panB(A159S) | | | | |
| ATCC 13032 pECCG117- | 0.8 | 0.7 | 0.8 | 0.77 |
| panB(A159Y) | | | | |
| ATCC 13032 pECCG117- | 2.2 | 2.3 | 2.2 | 2.23 |
| panB(A159C) | | | | |
| ATCC 13032 pECCG117- | 1.2 | 1.3 | 1.3 | 1.27 |
| panB(A159P) | | | | |
| ATCC 13032 pECCG117- | 0.7 | 0.8 | 0.7 | 0.73 |
| panB(A159H) | | | | |
| ATCC 13032 pECCG117- | 2.8 | 3.1 | 2.9 | 2.93 |
| panB(A159L) | | | | |
| ATCC 13032 pECCG117- | 2.5 | 2.7 | 2.6 | 2.6 |
| panB(A159I) | | | | |
| ATCC 13032 pECCG117- | 2.1 | 2.1 | 2.3 | 2.17 |
| panB(A159T) | | | | |
| ATCC 13032 pECCG117- | 0.9 | 0.6 | 0.7 | 0.73 |
| panB(A159K) | | | | |
| ATCC 13032 pECCG117- | 2.6 | 2.7 | 2.4 | 2.57 |
| panB(A159V) | | | | |
| ATCC 13032 pECCG117- | 1.9 | 1.9 | 2.2 | 2 |
| panB(A159M) | | | | |
| ATCC 13032 pECCG117- | 1.3 | 1.1 | 1.2 | 1.2 |
| panB(A159D) | | | | |
| ATCC 13032 pECCG117- | 0.8 | 0.8 | 0.7 | 0.77 |
| panB(A159E) | | | | |
| ATCC 13032 pECCG117- | 1.2 | 1.5 | 1.6 | 1.43 |
| panB(A159N) | | | | |
| ATCC 13032 pECCG117- | 1.2 | 1.1 | 1.2 | 1.17 |
| panB(A159Q) | | | | |
| ATCC 13032 pECCG117-panB(WT) | 0.7 | 0.7 | 0.7 | 0.7 |

In addition, as could be confirmed in Table 11 above, the wild type Corynebacterium glutamicum ATCC 13032 strain did not produce pantothenic acid, whereas all the 19 kinds of mutant strains produced above showed the pantothenic acid production ability, and among them, the mutant strain in which the amino acid corresponding to the 159th residue of the wild type panB (SEQ ID NO: 37) was mutated into arginine (R), serine(S), tyrosine (Y), cysteine (C), proline (P), histidine (H), leucine (L), isoleucine (I), threonine (T), lysine (K), valine (V), methionine (M), aspartic acid (D), glutamic acid (E), asparagine (N), or glutamine (Q), produced pantothenic acid at a high level, compared to the ATCC 13032 pECCG117-panB (WT) strain comprising the wild type panB. As a result, it could be confirmed that the amino acid residue corresponding to the 159th residue of panB (SEQ ID NO: 37) was the important position for pantothenic acid production, and it could be confirmed that when the amino acid at this position was substituted with various amino acids different from the original one, the production ability of pantothenic acid was further increased.

The ATCC 13032 ΔpanB pECCG117-panB (A159L) strain confirmed as having the most excellent production ability of pantothenic acid in the present example was named was CV03-5002 and was deposited to Korean Culture Center of Microorganisms located in Hongje-dong, Seodaemun-gu, Seoul, Korea on Apr. 13, 2021 and was given an accession number of KCCM12973P.

### Example 8. Production and Evaluation of Mutant 3-Methyl-2Ketobutanoate Hydroxymethyltransferase with Enhanced Pantothenic Acid Production Ability

It was confirmed whether there was an additional activity improvement by combining the 3-methyl-2ketobutanoate hydroxymethyltransferase G116A variant and the 3-methyl-2ketobutanoate hydroxymethyltransferase A159L variant of which effect was confirmed in Example 7.

To produce a vector comprising the G116A and A159L variants, a vector comprising both G116A and A159L was produced using primers of SEQ ID NOs: 74, 126 and SEQ ID NOs: 77, 127 using pECCG117-panB (A159L) produced in Example 2 as a template in the same way as described in Example 2. The produced mutant plasmid pECCG117-panB (G116A, A159L) was introduced into the wild type Corynebacterium glutamicum ATCC13032 strain by an electric pulse method, and then was smeared into a selective medium containing kanamycin 25 mg/L to obtain each transformed strain. Then, by progressing flask evaluation by the same method as Example 2, the production ability of the obtained pantothenic acid was measured. The obtained result was shown in Table 12 below.

TABLE 12
Pantothenic acid production ability of mutant strain comprising G116A and A159L

| | Pantothenic acid (g/L) | | | |
|---|---|---|---|---|
| | Arrangement | Arrangement | Arrangement | |
| Strain | 1 | 2 | 3 | Average |
| ATCC 13032 | 0.0 | 0.0 | 0.0 | 0 |
| ATCC 13032 pECCG117-panB(WT) | 0.7 | 0.7 | 0.6 | 0.67 |
| ATCC 13032 pECCG117- | 2.9 | 2.8 | 3.1 | 2.93 |
| panB(A159L) | | | | |
| ATCC 13032 pECCG117- | 2.2 | 2.4 | 2.1 | 2.23 |
| panB(G116A) | | | | |
| ATCC 13032 pECCG117- | 3.9 | 3.7 | 3.7 | 3.77 |
| panB(G116A, A159L) | | | | |

As could be confirmed in Table 12 above, the ATCC 13032 strain did not produce pantothenic acid, whereas the mutant strain comprising all the amino acid mutation corresponding to the 116th residue of panB and the amino acid mutation corresponding to the 159th residue produced pantothenic acid at a higher level compared to the ATCC 13032 pECCG117-panB (WT) strain comprising the wild type panB, and produced pantothenic acid at a higher level even than the mutant strains comprising an A159L or G116A mutation. As a result, it could be confirmed that the amino acids corresponding to the 116th and 159th residues of panB were important positions for pantothenic acid production, and it could be confirmed that when the amino acids at these positions were substituted with amino acids different from the original one, the production ability of pantothenic acid was further increased.

From the above description, those skilled in the art to which the present application pertains will be able to understand that the present application may be embodied in other specific forms without changing the technical spirit or essential characteristics thereof. In this regard, it should be understood that the examples described above are illustrative in all respects and not limitative. The scope of the present application should be construed as including all changes or modifications derived from the meaning and scope of the claims to be described below and equivalent concepts rather than the detailed description above, in the scope of the present application.

### [Accession Number]

Depository authority name: Korean Culture Center of Microorganisms
Accession number: KCCM12744P
Accession date: 20200608
Depository authority name: Korean Culture Center of Microorganisms
Accession number: KCCM12973P
Accession date: 20210413

1. A polypeptide, in which an amino acid corresponding to the 159th residue from the N-terminus in the amino acid sequence of SEQ ID NO: 37 is substituted with another amino acid.

2. The polypeptide according to claim 1, wherein the amino acid corresponding to the 159th residue is substituted with arginine (R), serine(S), tyrosine (Y), cysteine (C), proline (P), histidine (H), leucine (L), isoleucine (I), threonine (T), lysine (K), valine (V), methionine (M), aspartic acid (D), glutamic acid (E), asparagine (N), or glutamine (Q).

3. The polypeptide according to claim 1, wherein the polypeptide comprises the amino acid sequence of any one SED ID NO selected from the group consisting of amino acid sequences of SEQ ID NO: 110 to SEQ ID NO: 125.

4. The polypeptide according to claim 1, in which
an amino acid corresponding to the 116th residue from the N-terminus in the amino acid sequence of SEQ ID NO: 37 is further substituted with another amino acid.

5. The polypeptide according to claim 4, wherein
the amino acid corresponding to the 116th residue from the N-terminus is substituted with alanine (A), asparagine (N), threonine (T), glutamic acid (E), serine(S), valine (V), isoleucine (I), leucine (L), aspartic acid (D), cysteine (C), glutamine (Q), or methionine (M).

6. The polypeptide according to claim 4, wherein the polypeptide comprises the amino acid sequence of SEQ ID NO: 128.

7. A polynucleotide encoding the polypeptide of claim 1.

8. (canceled)

9. A microorganism producing pantothenic acid or pantoic acid,
wherein the microorganism comprises the polypeptide according to claim 1, or a polynucleotide encoding the polypeptide.

10-13. (canceled)

14. The microorganism according to claim 9, wherein the microorganism is a genus Corynebacterium microorganism or a genus Escherichia microorganism.

15. The microorganism according to claim 14, wherein the genus Corynebacterium microorganism is Corynebacterium glutamicum.

16. (canceled)

17. A method for preparation of pantothenic acid or pantoic acid, comprising culturing the microorganism of claim 9 in a medium.

18. The method for preparation of pantothenic acid or pantoic acid of claim 17, further comprising recovering pantothenic acid or pantoic acid from the cultured microorganism, medium or both of them, after the culturing.

19. A polynucleotide encoding the polypeptide of claim 4.

20. A microorganism producing pantothenic acid or pantoic acid,
wherein the microorganism comprises the polypeptide according to claim 4, or a polynucleotide encoding the polypeptide.
